(19) United States
(12) Patent Application Publication
(10) Pub. No.: US 2016/0367220 A1
(43) Pub. Date: Dec. 22, 2016

(54) ULTRASOUND DEVICE AND METHOD FOR ESTIMATING TISSUE STIFFNESS

(71) Applicants: Vanderbilt University, Nashville, TN (US); Duke University, Durham, NC (US)

(72) Inventors: Brett C. BYRAM, Brentwood, TN (US); Kristy M. WALSH, Nashville, TN (US); Douglas M. DUMONT, Nashville, TN (US); Mark L. PALMERI, Durham, NC (US)

(21) Appl. No.: 15/189,919

(22) Filed: Jun. 22, 2016

Related U.S. Application Data
(60) Provisional application No. 62/183,000, filed on Jun. 22, 2015.

(51) Int. Cl. A61B 8/08; A61B 8/00

(52) U.S. Cl. CPC A61B 8/485; A61B 8/4483; A61B 8/5207; A61B 8/587; A61B 8/0808; A61B 8/0883; A61B 8/0875; A61B 8/0858; A61B 8/54; A61B 8/5215

(57) ABSTRACT

An ultrasound device and method for calculating an estimated tissue stiffness based on peak on-axis tissue displacement propagating along the axis of an acoustic radiation force (ARF) excitation region are disclosed. Embodiments estimate stiffness by measuring time-to-peak tissue displacement directly along the acoustic radiation force axis. Embodiments achieve a highly accurate tissue stiffness estimate with minimal stiffness estimation error via use of an ultrasound device having less hardware complexity and which results in reduced sequencing complexity.

### CROSS REFERENCE TO RELATED APPLICATION(S)

This application claims priority from U.S. Provisional Patent Application No. 62/183,000, filed on Jun. 22, 2015, which is hereby incorporated herein by reference in its entirety.

### STATEMENT REGARDING FEDERALLY SPONSORED RESEARCH OR DEVELOPMENT

This invention was made with U.S. Government support under Grant No. R01 EB002132 awarded by the National Institutes of Health. The government has certain rights in the invention.

### FIELD OF THE INVENTION

Embodiments are in the field of ultrasound devices. More particularly, embodiments disclosed herein relate to ultrasound devices and methods for calculating an estimated tissue stiffness which, inter alia, foster a highly accurate tissue stiffness estimate with minimal stiffness estimation error via use of an ultrasound device having less hardware complexity and which results in reduced sequencing complexity.

### BACKGROUND OF THE INVENTION

Conventional ultrasound devices estimate tissue stiffness by using an array of transducers, each with its own additional circuit elements, to measure the shear wave velocity at a distance away from the acoustic radiation force (ARF) axis.

By measuring the time-to-peak displacement along the ARF axis, embodiments of the ultrasound device and corresponding method described herein are able to estimate tissue stiffness using a single transducer and fewer additional circuit elements than conventional systems. It has been found that on-axis time-to-peak displacement measurements along the ARF axis had too high of a variance to make feasible tissue stiffness measurements. The embodiments described herein reduce the stiffness estimation error by applying an adaptive displacement estimator such as a Bayesian estimator.

Thus, it is desirable to provide an ultrasound device for calculating an estimated tissue stiffness and method for calculating an estimated tissue stiffness that are able to overcome the above disadvantages.

Advantages of the present invention will become more fully apparent from the detailed description of the invention hereinbelow.

### SUMMARY OF THE INVENTION

Embodiments are directed to an ultrasound device for calculating an estimated tissue stiffness based on peak on-axis tissue displacement propagating along the axis of an acoustic radiation force (ARF) excitation region. The device comprises: a transducer that outputs acoustic radiation to induce ARF along the axis to displace the tissue; a receiver that receives, along the axis, acoustic radiation reflected by the displaced tissue and which is based on the ARF-induced tissue displacement, wherein the reflected acoustic radiation corresponds to a tissue response to the tissue displacement; and an analysis system. The analysis system comprises: an adaptive displacement estimator that estimates the displacement of the tissue at a plurality of depths per each select location along the axis; a timing system that determines (or identifies) a time for the tissue to achieve peak displacement; and a stiffness estimator that determines the estimated stiffness of the tissue based on the time for the tissue to achieve the peak displacement.

In an embodiment, the timing system determines (or identifies) the time for the tissue to achieve peak displacement using the estimated displacement of the tissue at the plurality of depths along the axis.

In an embodiment, the adaptive displacement estimator determines a probability of the estimated displacement given the tissue response. The adaptive displacement estimator may determine the sum square difference between the tissue response and a prior acoustic radiation reference signal weighted by local estimates of displacement estimation quality and prior information about acoustic radiation displacement behavior of the tissue at the plurality of depths along the axis.

In an embodiment, the adaptive displacement estimator is a Bayesian displacement estimator.

In an embodiment, the time determined (or identified) by the timing system is an estimated time.

In an embodiment, the stiffness estimator comprises a lookup table, wherein the estimated stiffness of the tissue is determined using the lookup table.

In an embodiment, the estimated stiffness of the tissue is determined by the stiffness estimator via a simulation or measurements from calibrated phantoms.

In an embodiment, the tissue is selected from the group consisting of liver, spleen, skin, heart, brain, muscle, and bone.

In an embodiment, the analysis system further comprises a motion filter that filters motion of the displacement of the tissue not due to the outputted acoustic radiation.

In an embodiment, the receiver is contained within the transducer.

In an embodiment, the ultrasound device comprises only a single transducer.

In an embodiment, the outputted acoustic radiation comprises long acoustic pulses and wherein the reflected acoustic radiation comprises short acoustic pulses which are shorter than the long acoustic pulses.

Embodiments are also directed to a method for calculating an estimated tissue stiffness based on peak on-axis tissue displacement propagating along the axis of an acoustic radiation force (ARF) excitation region. The method comprises: outputting acoustic radiation to induce ARF along the axis to displace the tissue; receiving, along the axis, acoustic radiation reflected by the displaced tissue and which is based on the ARF-induced tissue displacement, wherein the reflected acoustic radiation corresponds to a tissue response to the tissue displacement; estimating, using an adaptive displacement estimator, the displacement of the tissue at a plurality of depths per each select location along the axis; determining a time for the tissue to achieve peak displacement; and determining the estimated stiffness of the tissue based on the time for the tissue to achieve the peak displacement.

In an embodiment, the determining of the time for the tissue to achieve peak displacement uses the estimated displacement of the tissue at the plurality of depths along the axis from the estimating step.

In an embodiment, the estimating step comprises determining, using the adaptive displacement estimator, a probability of the estimated displacement given the tissue response. The estimating step may further comprise determining, using the adaptive displacement estimator, the sum square difference between the tissue response and a prior acoustic radiation reference signal weighted by local estimates of displacement estimation quality and prior information about acoustic radiation displacement behavior of the tissue at the plurality of depths along the axis.

In an embodiment, the adaptive displacement estimator is a Bayesian displacement estimator.

In an embodiment, the time determined by the determining the time step is an estimated time.

In an embodiment, the determining of the estimated stiffness of the tissue comprises using a lookup table.

In an embodiment, the determining of the estimated stiffness of the tissue comprises using a simulation or measurements from calibrated phantoms.

In an embodiment, the tissue is selected from the group consisting of liver, spleen, skin, heart, brain, muscle, and bone.

In an embodiment, the method further comprises filtering motion of the displacement of the tissue not due to the outputted acoustic radiation.

In an embodiment, the outputting and receiving steps are performed by a single transducer.

In an embodiment, the outputted acoustic radiation comprises long acoustic pulses and wherein the reflected acoustic radiation comprises short acoustic pulses which are shorter than the long acoustic pulses.

### BRIEF DESCRIPTION OF THE DRAWINGS

The detailed description will refer to the following drawings, wherein like reference numerals refer to like elements, and wherein:

FIG. 1 is a block diagram illustrating an embodiment of hardware and software components of the on-axis stiffness estimation device;

FIG. 2 is a diagram illustrating an acoustic radiation force impulse (ARFI) simulation and a comparison of on-axis displacements and shear wave displacements;

FIGS. 3A-3C are diagrams illustrating ARFI displacement;

FIG. 4 is a diagram illustrating simulated ARFI displacement data used to make a look-up table;

FIG. 5 is a block diagram illustrating an on-axis stiffness estimation algorithm;

FIG. 6 is a plot illustrating a simulated test case;

FIG. 7 is a plot illustrating the mean square error for both normalized cross-correlation and Bayesian displacement estimation methods;

FIG. 8 is a plot illustrating bias in stiffness estimation when using different attenuations;

FIGS. 9A and 9B are a plot comparison illustrating the mean square error and bias results of the look-up tables with different stiffness sampling;

FIG. 10 is a plot illustrating the root mean square error of the shear modulus estimated using either the Bayesian displacement estimator or the NCC-derived estimator;

FIGS. 11A and 11B illustrate Bland Altman plots of shear moduli estimated from the SWS method and the on-axis method using Bayes and NCC;

FIG. 12 is a plot illustrating data used to make a look-up table using time-to-peak and depth to then extract a stiffness estimate;

FIGS. 13A and 13B are plots illustrating NCC vs. Bayesian estimation;

FIG. 14 is a chart illustrating simulation parameters for FEM look-up tables;

FIG. 15 is a plot illustrating simulation results from 20 realizations at Young's modulus of 8 kPa;

FIGS. 16A and 16B are a plot comparison illustrating phantom motion filtering;

FIGS. 17A and 17B are another plot comparison illustrating no motion filter vs. quadratic motion filter for phantoms;

FIG. 18 is a diagram illustrating application of Bayes Theorem to Displacement;

FIG. 19 is a diagram illustrating maximization of posterior PDF;

FIG. 20 is a diagram illustrating the noise term of the likelihood function;

FIG. 21 is a diagram illustrating shear modulus in a plate-like media;

FIGS. 22A-22C are a plot comparison illustrating time-to-peak velocity including simulations of phantoms using a look-up table, and with raw and smooth filtering applied;

FIGS. 23A-23B are plots illustrating look-up table simulation in FIG. 22 in comparison with an on-axis time-to-peak displacement;

FIGS. 24A and 24B are a plot comparison illustrating results of Bayes vs. NCC estimations with motion filtering included;

FIG. 25 is a diagram illustrating how error from an embodiment of an on-axis method is within the same range as SWEI error; and

FIG. 26 is a flowchart illustrating an embodiment of a method for calculating an estimated tissue stiffness based on peak on-axis tissue displacement propagating along the axis of an acoustic radiation force (ARF) excitation region.

### DETAILED DESCRIPTION OF THE INVENTION

It is to be understood that the figures and descriptions of the present invention may have been simplified to illustrate elements that are relevant for a clear understanding of the present invention, while eliminating, for purposes of clarity, other elements found in a typical ultrasound device, typical method of using an ultrasound device, or typical method of calculating estimated tissue stiffness using an ultrasound device. Those of ordinary skill in the art will recognize that other elements may be desirable and/or required in order to implement the present invention. However, because such elements are well known in the art, and because they do not facilitate a better understanding of the present invention, a discussion of such elements is not provided herein. It is also to be understood that the drawings included herewith only provide diagrammatic representations of the presently preferred structures of the present invention and that structures falling within the scope of the present invention may include structures different than those shown in the drawings. Reference will now be made to the drawings wherein like structures are provided with like reference designations.

Before explaining at least one embodiment in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting.

It should further be understood that any one of the described features may be used separately or in combination with other features. Other invented devices, systems, methods, features, and advantages will be or become apparent to one with skill in the art upon examining the drawings and the detailed description herein. It is intended that all such additional devices, systems, methods, features, and advantages be protected by the accompanying claims.

There are potentially many different target areas/applications for the method/system described in this disclosure, although the liver is the focus herein for purposes of explanation only.

Embodiments described herein include an ultrasound device/method for estimating tissue stiffness by outputting acoustic radiation (e.g., by a single transducer) along an axis to displace the tissue, receiving, along the axis, acoustic radiation reflected by the tissue and which is based on the ARF-induced tissue displacement, wherein the reflected acoustic radiation corresponds to a tissue response to the tissue displacement, using an adaptive displacement estimator such as a Bayesian estimator to estimate the displacement of the tissue at a plurality of depths per each select location along the axis, estimating a time for the tissue to achieve peak displacement, and estimating the stiffness of the tissue based on the time for the tissue to achieve the peak displacement (e.g., using a lookup table, a simulation or measurements from calibrated phantoms). The Bayesian estimator may determine the probability of the estimated displacement given the tissue response. Further, the Bayesian estimator may estimate the displacement by determining the sum square difference between the tissue response and a prior acoustic radiation reference signal weighted by local estimates of displacement estimation quality and prior information about acoustic radiation displacement behavior of the tissue at the plurality of depths along the axis. Unlike other stiffness estimation techniques, the present invention uses RF data within the region of acoustic radiation force (ARF) excitation which will likely result in minimized hardware and processing required to estimate stiffness.

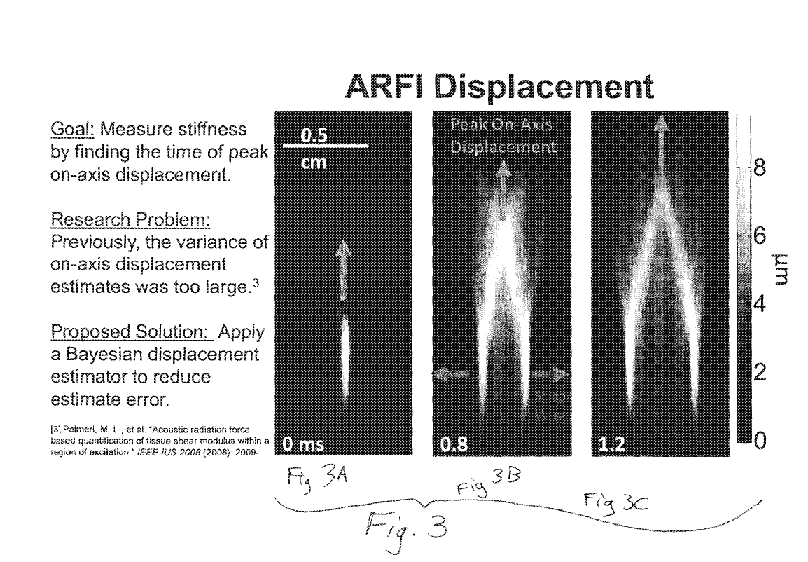

In prior shear wave elasticity imaging, stiffness can be estimated by measuring shear wave velocity at locations away from the ARF axis. Instead, embodiments herein estimate stiffness by measuring the time-to-peak displacement directly along the ARF axis, which reduces hardware and sequencing complexity (FIG. 3B). Using a look-up table generated by an FEM model, we have shown previously in simulation results that an advanced displacement estimator reduces variability in the final stiffness estimate. The on-axis approach has been tested in 15 phantoms.

In the present disclosure, it is assumed that the phantoms are homogeneous, isotropic, and linearly elastic, thus time-to-peak displacement is directly proportional to shear wave speed. Since shear wave speed is directly related to shear stiffness, we create a stiffness look-up table of the time-to-peak displacement as a function of depth. We generated look-up tables using a 3D FEM model coupled to Field II simulations and the selected displacement estimation method. We simulated time-to-peak look-up tables for shear moduli from 1-15 kPa and attenuation of 0.7 dB/cm-MHz. We used a CH4-1 probe with excitation focal depth of 4.9 cm, transmit F/#2, and transmit frequency of 3.08 MHz. Both normalized cross correlation (NCC) and Bayesian displacement estimators were evaluated. We applied a quadratic motion filter to the data. To evaluate the error of the on-axis method as compared to traditional shear wave methods, we computed a robust lateral time-of-flight shear wave speed using a Radon sum transformation (LATSUM) and converted to a shear modulus for each phantom.

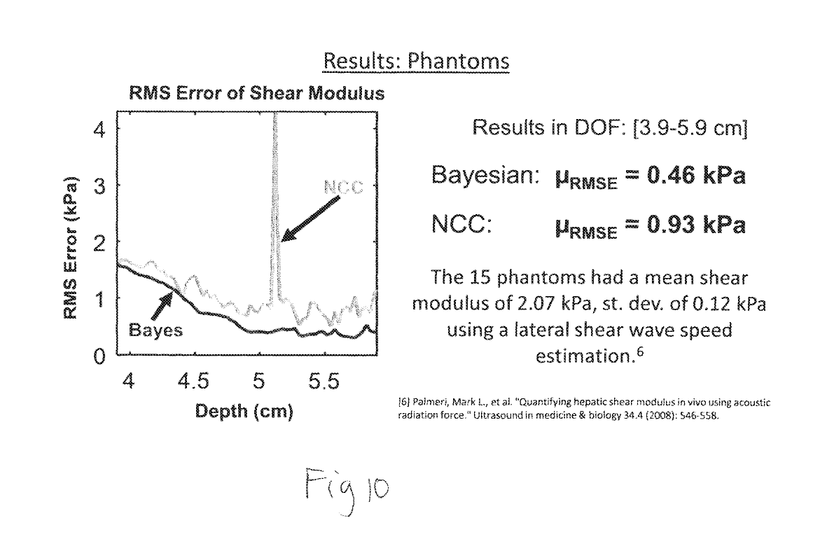

The 15 phantoms had a mean shear modulus of 2.07 kPa and standard deviation of 0.12 kPa. We took the root mean square error of the shear modulus estimated using either the Bayesian displacement estimator or the NCC-derived estimator (FIG. 10). In the depth of field, the median RMSE of shear modulus for the Bayesian estimator was 0.46 kPa and 0.93 kPa for NCC. In the phantom experiments, stiffness was not known for the phantoms. The error was computed between our results and a robust shear wave speed method measured at lateral locations. RMS Error for Bayes is below NCC at all depths within the depth of field.

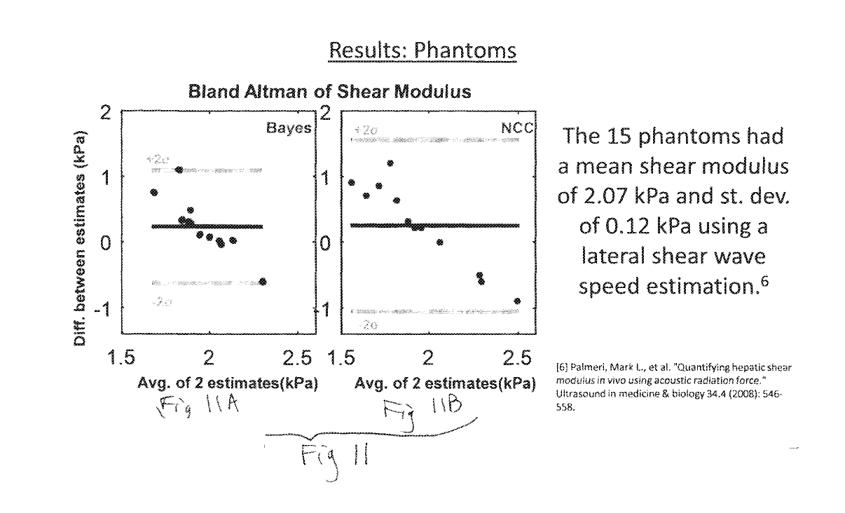

FIGS. 11A and 11B illustrate Bland Altman plots of shear moduli estimated from the SWS method and our on-axis method using Bayes and NCC. The Bayes results show more agreement with the SWS results than NCC. These phantom results show that on-axis methods coupled with a Bayesian displacement estimator produce stiffness estimates comparable to laterally offset shear wave methods. Specifically, FIGS. 11A and 11B show Bland Altman plots of shear moduli estimated from the LATSUM method and our on-axis method using Bayes and NCC. The Bayes results show more agreement with the LATSUM results than NCC. These results in phantoms show that the on-axis method produces stiffness estimates comparable to Lateral shear wave methods when using a Bayesian displacement estimator rather than NCC.

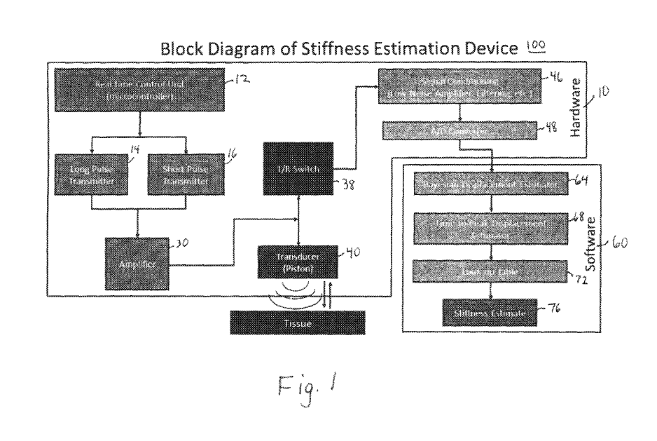

With reference to FIG. 1, shown is a block diagram illustrating an embodiment of hardware components 10 and software components 60 of the on-axis stiffness estimation device 100. In the hardware components 10 section, we generate short pulses for estimating motion and long pulses for generating acoustic radiation force induced displacements. A real-time controller (microcontroller) 12 synchronizes the long pulse transmitter 14 and the short pulse transmitter 16 and toggles the T/R switch 38 to protect the receive circuitry. The transmit pulses from the long pulse transmitter 14 and the short pulse transmitter 16 are amplified via amplifier 30 and sent through the T/R switch 38 to the transducer 40. In an embodiment, the device may comprise only a single piston transducer 40. After transmitting, the T/R switch 38 allows the low voltage receive signal to pass through to the receive circuitry (signal conditioning 46). The receive circuitry for signal conditioning 46 includes standard components such as a low noise amplifier, a variable gain amplifier and bandpass filters. After signal conditioning 46, the signal is sampled by the A/D converter 48.

The sampled data is passed along to the software component 60 portion of the device 100. In the software component 60, we take the sampled radio-frequency (RF) signal and compute displacement using an adaptive displacement estimator such as a Bayesian displacement estimator 64. Then, we find the time-to-peak displacement using a time-to-peak displacement estimator 68 at each depth. We use the estimated time-to-peak displacement and depth as inputs into the look-up table 72 generated by simulations. The output of the look-up table is a quantitative estimate of tissue stiffness 76. The software component 60 may be physically separate from the hardware component 10 enclosure. Alternatively, the software component 60 may be contained within the hardware component 10 enclosure.

The adaptive displacement estimator is an estimator of the type that utilizes local estimates of displacement estimate quality combined with prior information of ARF displacement characteristics, which contrasts to other displacement estimators (such as normalized cross correlation (NCC)) commonly used for shear wave and other ARF techniques that do not weight the displacements based on local quality and do not consider prior information about ARF displacement behavior of the tissue.

In traditional shear wave elasticity imaging (SWEI), shear wave velocity is measured away from the acoustic radiation force (ARF) axis. Instead, we measure the time-to-peak displacement of tissue directly along the ARF axis. Measuring displacements along this axis rather than off-axis simplifies hardware required for quantifying tissue stiffness. Previously this method has been demonstrated, but the measurement variance was too high for practical feasibility. To reduce stiffness estimation error, we apply our Bayesian displacement estimator.

To evaluate the Bayesian estimator, we used 3D finite element analysis to model soft tissue response to the acoustic radiation force and Field II to simulate the radio-frequency (RF) data of the tissue response. The Bayesian displacement estimator is applied to RF data to improve tissue displacement estimates, which then improves time-to-peak displacement estimates and the final stiffness estimate. Time-to-peak displacement is proportional to shear wave speed if we assume the medium is linear, elastic, and isotropic. Here, shear wave speed is directly related to shear stiffness, and we create look-up tables to estimate stiffness using time-to-peak displacement as a function of depth. We modeled an L12-5 50 mm linear transducer with a transmit frequency of 7.8 MHz, 2 cm focus, and push F/2.5. The average displacement data from 20 speckle realizations of each tissue stiffness were used to generate the stiffness look-up tables. Our Bayesian displacement estimator had lower mean square error (MSE) in stiffness estimates compared to using a traditional Normalized Cross-Correlation (NCC) estimator.

In linear, isotropic, elastic tissues, the shear wave speed (c) is directly proportional to the stiffness in a tissue with constant density:

$$c = \sqrt{\frac{μ}{ρ}} \qquad ( 1 )$$

where μ is the shear stiffness and ρ is the density.

Shear wave elasticity imaging (SWEI) uses an ultrasound beam to induce tissue displacement, then measures the speed of the shear waves propagating outward at lateral locations. By equation (1), you can determine the stiffness by measuring the shear wave speed. In traditional SWEI, the shear wave propagating outward is measured at locations shown by the horizontal arrows in FIG. 3.

Previously, a method was developed to measure stiffness without needing lateral information, but only using the on-axis displacement data. As the shear waves propagate outward, an interference displacement propagates upwards towards the transducer, shown by the vertical arrows in FIG. 3. Alternatively to SWEI, we measure the time of displacement along this axis. By calculating the time of peak on-axis displacement, we can correlate this to the shear wave speed to measure stiffness. Time-to-peak (TTP) displacements are depth dependent and also a function of the spatial distribution of the excitation. This motivates using a look-up table approach to describe the time-to-peak at each depth for a certain excitation distribution. Here, we will estimate stiffness in Young's Modulus, E=3μ, for an incompressible medium.

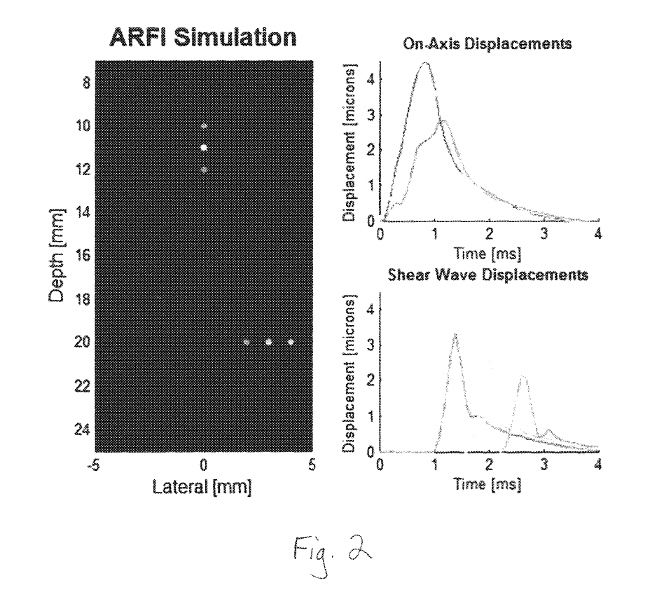

FIG. 2 is a diagram illustrating an ARFI simulation and a comparison of on-axis displacements and shear wave displacements. Shown is a simulation of an acoustic radiation force excitation. The excitation region experiences displacement from the acoustic pulse. As the shear waves propagate away from the excitation, there is an interference pattern that propagates up towards the transducer in the on-axis region. Shear wave elasticity measures the displacements at lateral locations. Embodiments herein only measure displacement in the on-axis region shown in the vertical arrows (see FIG. 3). By assuming the medium is linear, elastic, and isotropic, we can relate the shear wave speed to a proportional time-to-peak displacement. An advantage of measuring displacements along this axis rather than off-axis is simplifying hardware required for quantifying tissue stiffness. Also, the on-axis method could better measure wave propagation in complicated media. This isn't as necessary in the liver, but could help in other applications.

FIG. 3 is a diagram illustrating ARFI displacement. ARFI Simulation is shown with propagation of displacements over time. The horizontal arrows in the second time frame show shear waves propagating laterally away from the ARF excitation region. The vertical arrows show the on-axis displacement propagating upwards along the axis of the ARF excitation region. This study measures the peak on-axis displacement along the on-axis line located along the vertical arrows. Unlike other stiffness estimation techniques, this only uses RF data within the region of acoustic radiation force (ARF) excitation. (vertical arrows). In traditional shear wave elasticity imaging (SWEI), shear wave velocity is measured away from (i.e., laterally from) the ARF axis via the horizontal arrows. Stiffness is measured by finding the time of peak on-axis displacement. Previously, on-axis stiffness estimation using ARF had a variance that was too large to make feasible stiffness estimates. The present invention reduces stiffness estimation error by applying an adaptive displacement estimator to on-axis displacement estimates.

This method is different than the Fibroscan® approach because it uses the displacements induced by an ARF excitation in the same orientation as SWEI.

A similar method of on-axis stiffness estimation, but without using an adaptive displacement estimator, had a high variance. To lower the variance produced in displacement estimation, we apply a Bayesian displacement estimator. We compare that to traditional Normalized Cross-Correlation displacement estimator to determine feasibility in this disclosure.

### Exemplary Methods

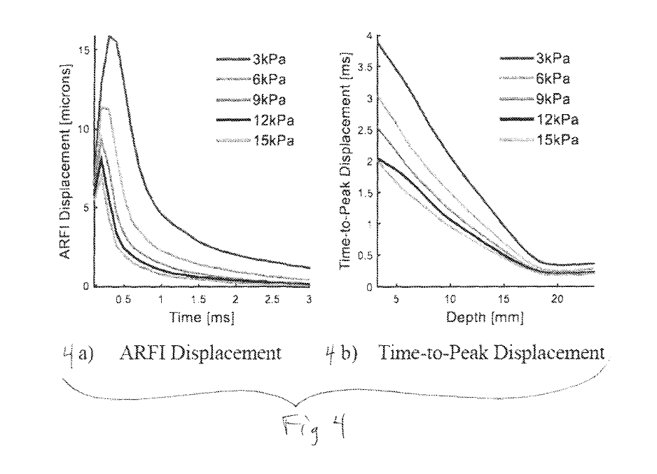

Simulated data were used to make look-up tables to estimate stiffness using time-to-peak displacement as a function of depth. FIG. 4 shows simulated ARFI displacement data used to make a look-up table. For a single depth shown in FIG. 4A, each stiffness has a different time to peak displacement. The time-to-peak displacement is shown in FIG. 4B for each stiffness as a function of depth. This data is used as a look-up table to extract stiffness information given a time-to-peak displacement value at a certain depth. In this study, the stiffness estimation method was tested using simulations of tissue responses. In FIG. 4, simulation data is used for look-up tables and averaged over 20 realizations for each stiffness. (FIG. 4A) ARFI displacements at a depth of 2 cm for each stiffness in Young's Modulus. (FIG. 4B) Time-to-peak displacements for each stiffness through depth.

A. Finite Element Method Simulations

Finite element simulations were used to generate data for varying stiffnesses. Tissue sample properties were simulated using a 1,500,000 element, 3D mesh with dimensions 0.5×1.0×3.0 cm. Field II was used to calculate the pressure field using the parameters in Table I. Simulated displacements were generated using LS-DYNA (Livermore Software Technology Corporation, Livermore, Calif.). The 3D volume of displacements was imported into Field II to displace point scatterers. We also used Field II to simulate on-axis ultrasonic tracking using the L12-5 probe configuration.

B. Stiffness Estimation Algorithm

To generate the look-up table, we used 20 Field II displacement simulations for each stiffness for Young's Moduli of 3, 6, 9, 12, and 15 kPa. After computing the on-axis lines of tracked RF data, we computed the ARF displacements using the Bayesian estimator.

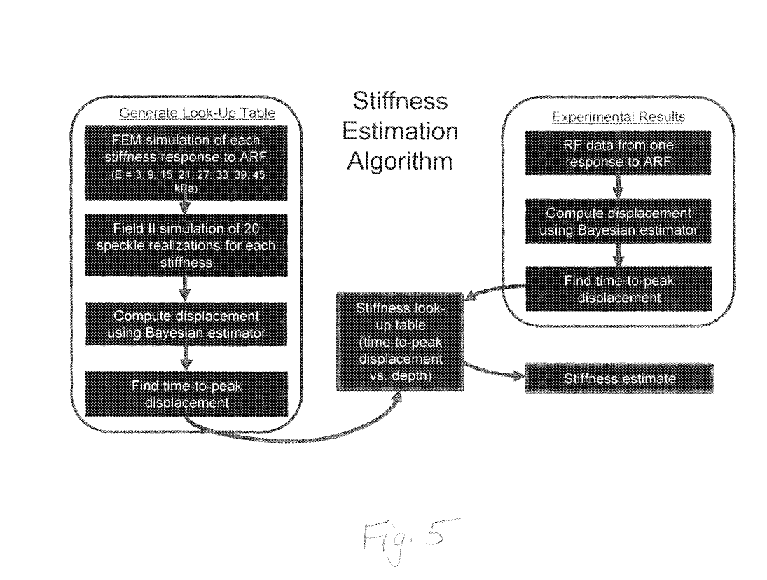

FIG. 5 is a block diagram illustrating an on-axis stiffness estimation algorithm. The left section shows the steps for generating the look-up table using simulations and the Bayesian displacement estimator. The right section shows the steps for an experiment simulation using the Bayesian displacement estimator and the look-up table to extract the stiffness estimate. To generate a lookup table, simulations are used. 3D finite element analysis is used to model soft tissue response to the acoustic radiation force for each stiffness. Field II simulation is used to simulate the radio-frequency data of the tissue response. This gives RF Data. The Bayesian displacement estimator is applied to RF data to improve tissue displacement estimates. The time-to-peak displacement is determined for each depth in the on-axis region. A look-up table is created to estimate stiffness using time-to-peak displacement as a function of depth.

The Bayesian displacement estimator uses the Bayes' Theorem to estimate a posterior probability density function (PDF) of a displacement estimate, τₖ, given the observed RF data, x, shown here as,

$${P}_{k} ( {τ}_{k} \vert x ) = \frac{{P}_{k} ( x \vert {τ}_{k} ) {P}_{k} ( {τ}_{k} )}{{P}_{k} ( x )} \qquad ( 2 )$$

where Pₖ(x|τₖ) is the likelihood function, Pₖ(τₖ) is the prior PDF, and Pₖ(x) is the marginal likelihood PDF. To find the displacements, τₖ, that maximizes the posterior PDF, we can describe the terms in (2) in the log-domain as,

$$\begin{matrix}ln ( {P}_{k} ( {τ}_{k} \vert x ) ) ∝ - \frac{1}{4 {σ}_{n}^{2}} {\sum }_{s = 0}^{M - 1} {( {r}_{k} [ s ] - {t}_{k} [ s \text{:} - {τ}_{k} ] )}^{2} … \\ - \frac{1}{p {λ}^{p}} {\sum }_{{k}_{v} \vert ∈ B} {w}_{j} \vert {τ}_{k} - {τ}_{j} {\vert}^{p}\end{matrix} \qquad ( 3 )$$

where the log likelihood is the sum-squared difference between the reference RF signal, rₖ[s], and the tracked RF signal delayed by −τₖ, tₖ[s;−τₖ], over the kernel length M The likelihood term is weighted by an adaptive noise term, σₙ², to account for the noise and decorrelation in both RF signals as shown here,

$${σ}_{n}^{2} = \frac{{P}_{RF}}{{SNR}_{ρ} + 1} \qquad ( 4 )$$

TABLE I
LOOK-UP TABLE SIMULATION PARAMETERS

| Parameter | Value | |
|---|---|---|
| Transducer | L12-5 50 | mm |
| Excitation Focal Depth | 2 | cm |
| Excitation F/# | 2.5 | |
| Tx. Center Frequency | 7.8 | MHz |
| Attenuation | 0.7 | dB/cm/MHz |

where P_{RF} is the power of the RF signal and SNR_{ρ} is derived from the peak correlation-coefficient estimate of the SNR shown here as,

$${SNR}_{ρ} = \frac{{ρ}_{max}}{1 - {ρ}_{max}} \qquad ( 5 )$$

where ρₘₐₓ is the peak of the normalized cross-correlation for the kernel k.

The prior PDF term is now represented in (3) as a weighted prior term where wⱼ weights adjacent displacement estimates, τⱼ, when calculating the current displacement estimate, τₖ, for a neighborhood B. This weighted prior term also has tuning parameters λ and p which scale the distribution of the prior PDF. In the Bayesian displacement estimator, we train the tuning parameters by minimizing the error in simulated displacement profiles.

To find the displacement estimates, we apply the maximum a posteriori principle to (3) for all N kernels in the dataset, shown as,

$${τ}^{\hat{\to }} = arg {max}_{\vec{τ}} {\sum }_{λ = ρ}^{N - 1} ln ( {P}_{k} ( {τ}_{k} \vert x ) ) \qquad ( 6 )$$

which maximizes the global, log-posterior probability. This gives us the vector of displacements.

Next, we find the time-to-peak displacement for each depth in the on-axis line. We do this for a range of stiffnesses. Then, we create a time-to-peak look-up table to estimate stiffness using time-to-peak as a function of depth.

In this study, we used experimental simulations. We followed the same finite element simulation as above. Then, we found the time-to-peak displacement for each depth in the test case and we extracted the stiffness estimate from the look-up table. This process is shown in FIG. 5.

C. Simulation Tests Using the Stiffness Look-Up Table

The first stiffness test used 20 simulated datasets at a Young's Modulus of 8 kPa.

Next, we tested the effect of using a different attenuation of the test cases as compared to the attenuation of the data used to make the look-up table. We used a test case simulated at an attenuation of 0.5 dB/cm/MHz and compared it to the test case simulated at the same attenuation of the look-up table, 0.7 dB/cm/MHz.

We also test the sampling of stiffnesses required for the look-up table. We removed the 9 kPa data from the look-up table and tested stiffness increments from 6 to 9 kPa.

### Results

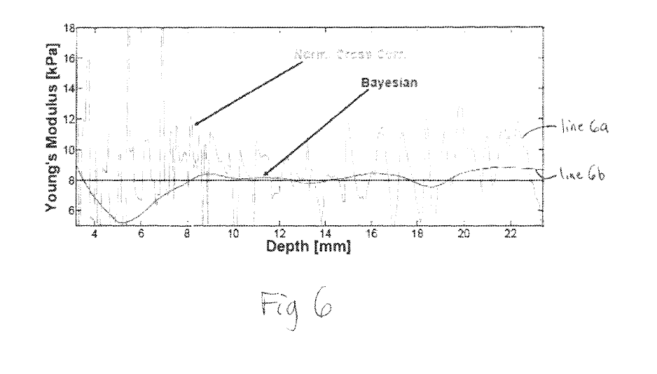

FIG. 6 shows the results for a test case of a Young's Modulus of 8 kPa. Line 6a used normalized cross-correlation to estimate displacements and line 6b uses the Bayesian displacement estimation. The black horizontal line is at 8 kPa. The Bayesian estimation is closer to the simulated stiffness, especially as the depth approaches the focus of 2 cm. FIG. 6 is a stiffness result for an E=8 kPa simulation. The black horizontal line shows the simulated stiffness of 8 kPa. Stiffness results using a Bayesian displacement estimator are shown line 6b and results using a normalized cross-correlation displacement estimator are shown by line 6a.

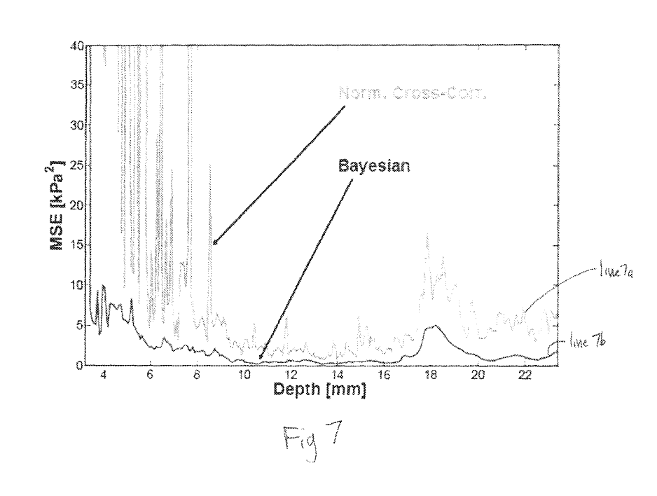

FIG. 7 shows the mean square error for both normalized cross-correlation and Bayesian displacement estimation methods. The stiffness estimate resulting from Bayesian displacement estimation has a lower mean square error at all depths. In FIG. 7, Mean Square Error (MSE) of 20 simulations at E=8 kPa. MSE results using a Bayesian displacement estimator are shown by line 7b and results using a normalized cross-correlation displacement estimator are shown by line 7a.

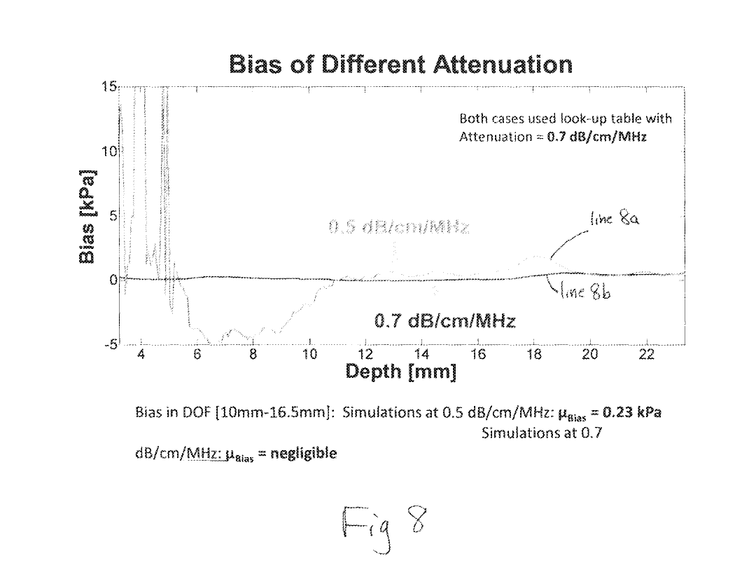

FIG. 8 shows the bias in stiffness estimation when using different attenuations. Both cases shown used a look-up table simulated at an attenuation of 0.7 dB/cm/MHz. FIG. 8 shows the bias result of a test case with the same attenuation as the look-up table via line 8b, and the result of a test case with a different attenuation of 0.5 dB/cm/MHz in red. When testing a different attenuation, there is a small bias near the focal depth, but outside of the focal depth, such as, in the near field, there is a substantial bias. FIG. 8 is a plot illustrating bias of different attenuations. Both cases used a look-up table with simulated attenuations of 0.7 dB/cm/MHz. Line 8b shows bias in the stiffness results when simulating tissue with the same attenuation as the look-up table, 0.7 dB/cm/MHz, and the line 8a shows the bias in the stiffness result when simulating at a different attenuation of 0.5 dB/cm/MHz.

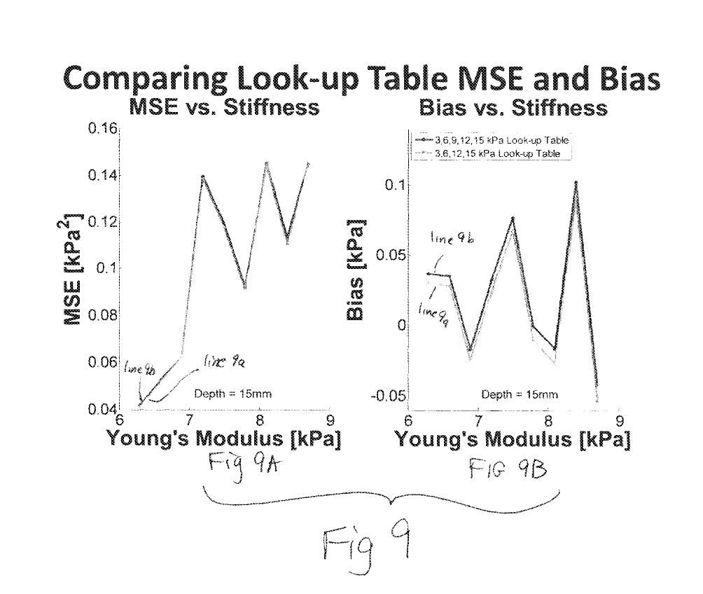

FIG. 9 shows the mean square error and bias results of the look-up tables with different stiffness sampling. When removing 9 kPa from the 3, 6, 9, 12, 15 kPa look-up table, the error and bias results are in the same range as including 9 kPa. The MSE and bias range is also small compared to the stiffness estimations and within the same range as SWEI error metrics. This shows the sampling of stiffnesses used to create the look-up table is sufficient. However, results could differ for stiffer tissues. FIG. 9 is a plot comparing mean square error and bias of different look-up tables. In both plots, line 9b shows results from a look-up table with stiffnesses 3, 6, 9, 12, and 15 kPa and line 9a shows results from a look-up table with stiffnesses 3, 6, 12, and 15 kPa.

The results show initial feasibility of the on-axis stiffness estimation using a Bayesian displacement estimator in simulations. As compared to normalized cross-correlation, the Bayesian displacement estimator improved the error of estimates at all depths. Estimating stiffness in different tissue attenuations may introduce large bias outside the focal depth, but near the focus could produce feasible estimates. The on-axis stiffness estimation could enable a way to produce quantitative stiffness estimation with simpler hardware.

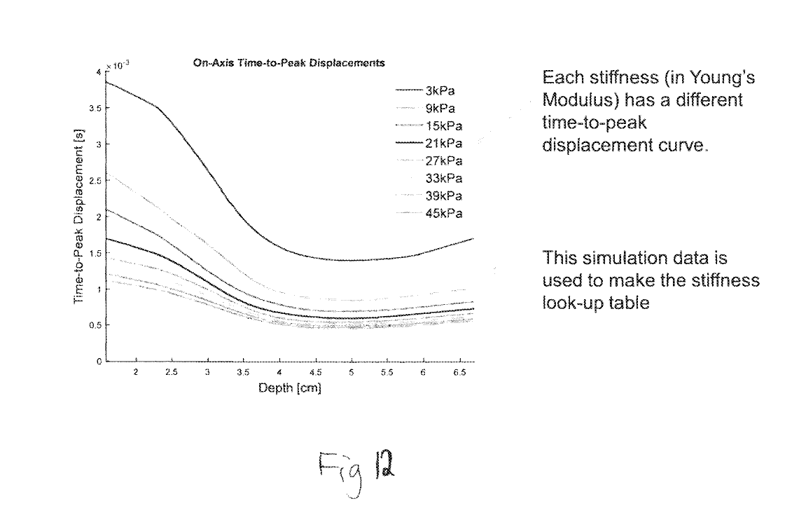

FIG. 12 is a plot illustrating data used to make a look-up table using time-to-peak and depth to then extract a stiffness estimate. Similar to measuring SWS, each tissue stiffness has a different on-axis time-to-peak displacement as a function of axial depth.

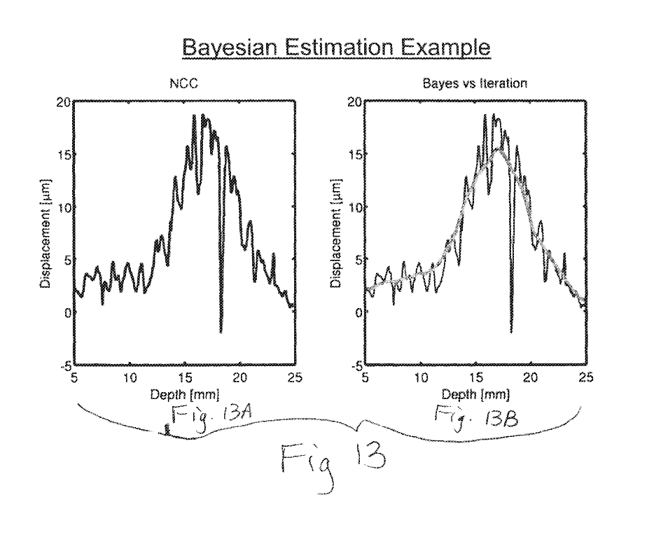

FIG. 13 is a plot illustrating NCC vs. Bayesian estimation. Bayesian estimation provides a “smoother” estimate than NCC and with better error metrics.

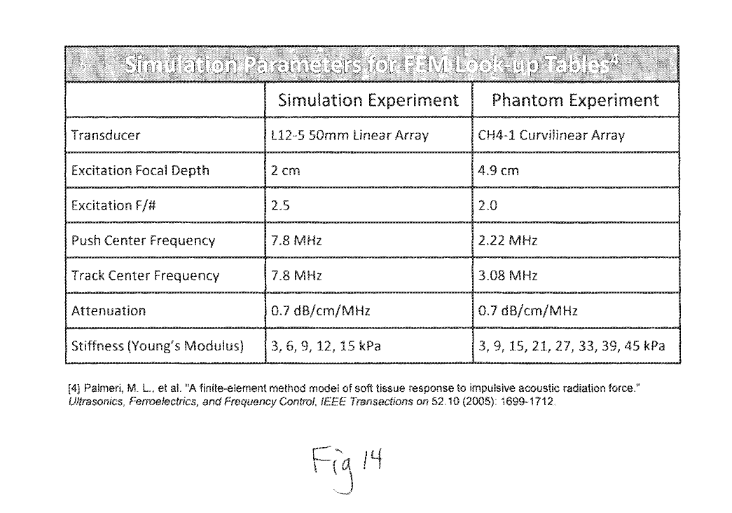

FIG. 14 is a chart illustrating simulation parameters for FEM look-up tables. Since, in an embodiment, the method uses a look-up table, each experiment/transducer configuration is modeled in FEM using these parameters. An experiment has been performed of stiffness in phantom data, which uses a simulated look-up table with these parameters (curvilinear probe, focus of 4.9 cm) to match the experimental data as closely as possible.

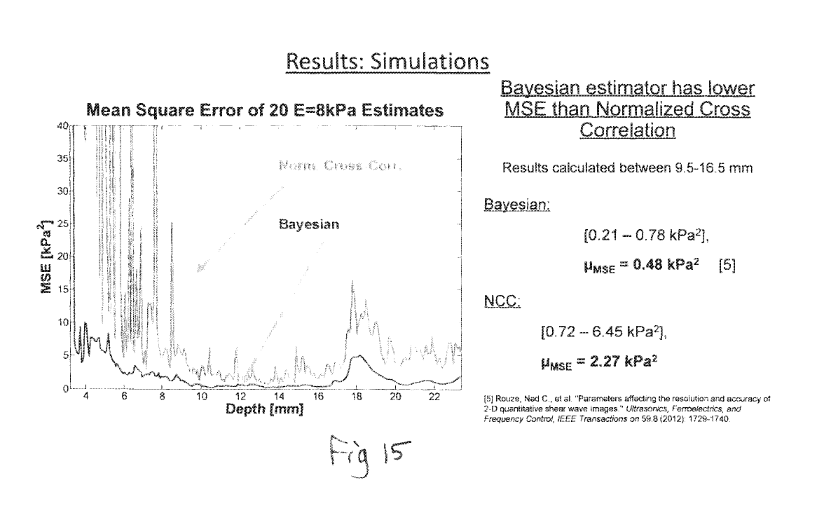

FIG. 15 is a plot illustrating simulation results from 20 realizations at Young's modulus of 8 kPa. Error is calculated based on the known stiffness of the simulations. We also compared our results with a Bayesian displacement estimator to a commonly used normalized cross correlation (NCC) method. The Bayesian estimator lowered the MSE at all depths and performed near the error of SWEI methods at depths between 9.5 and 16.5 mm.

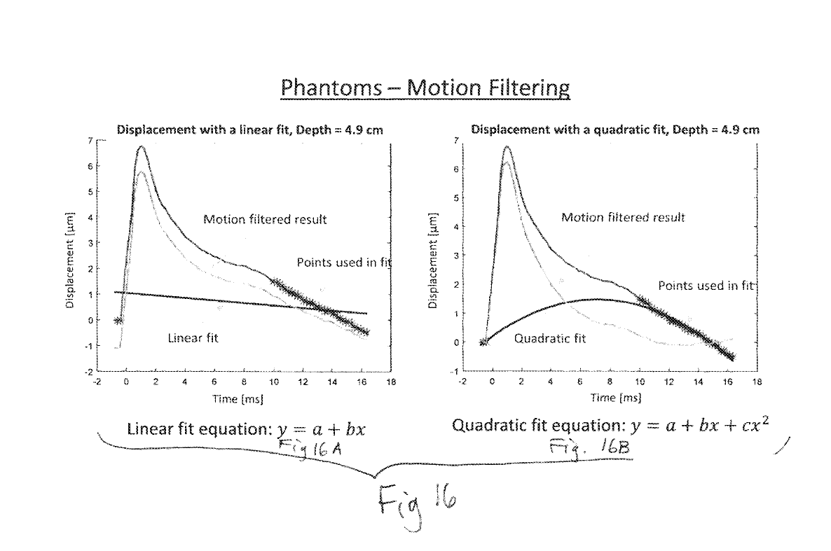

FIG. 16 is a plot comparison illustrating phantom motion filtering. Since real phantom data is used, motion must be accounted for. Motion filters have been applied (e.g., linear, quadratic, and cubic (not shown)). The filter has been applied to each ARFI displacement curve at each depth. Shown here is a depth at the focus of 4.9 cm. 2 points are used before the push and all the points after the tissue recovered from the displacement can fit the respective curves to the data and subtracted the resulting fit to remove motion.

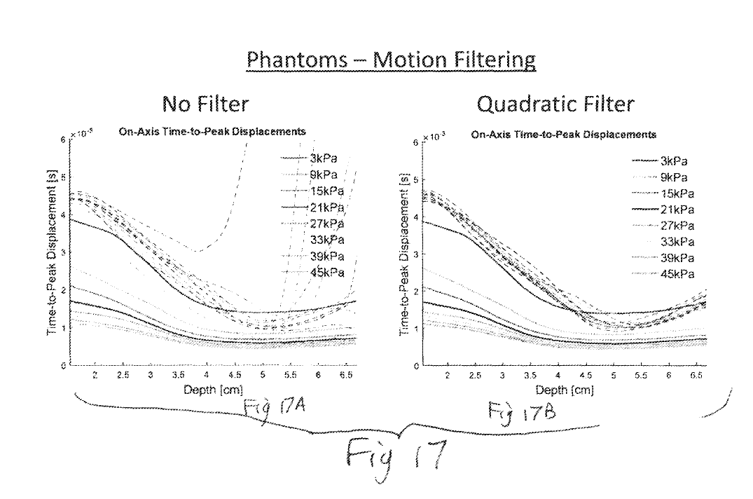

FIG. 17 is another plot comparison illustrating no motion filter vs. quadratic motion filter for a phantoms. The TTP curves for the look-up table are shown by the solid lines and each of the 15 phantoms are shown by the dotted lines. The phantoms were all around 6 kPa in Young's modulus and we determined that the quadratic filter performed the best.

It has been concluded that in phantoms, the Bayesian Displacement Estimator reduced the RMSE compared to a Normalized Cross-Correlation by 0.47 kPa within the depth of field. Also, the on-axis stiffness estimation with a Bayesian displacement estimator is within the error range of Shear Wave Speed (SWS) Time-of-Flight-based methods. Embodiments herein show feasibility in phantoms. Embodiments herein may be applied to in vivo data of patients with liver fibrosis stages 1-4.

An advantage of embodiments of the on-axis method over shear wave is that the on-axis method minimizes or makes negligible the effect of complicated wave propagation along the skin. The following are exemplary uses of embodiments described herein:
Staging sclerotic cutaneous skin diseases
   Morphea is a disorder characterized by excessive collagen deposition leading to thickening of the dermis, subcutaneous tissues, or both.
   Sclerotic graft versus host disease (GVHD) is an immune disorder in which the transplanted donor T cells recognize transplant recipient cells as foreign and kill them, including those in the skin. Sclerosis is a distinct clinical phenotype of chronic GVHD.
Radiation induced skin fibrosis

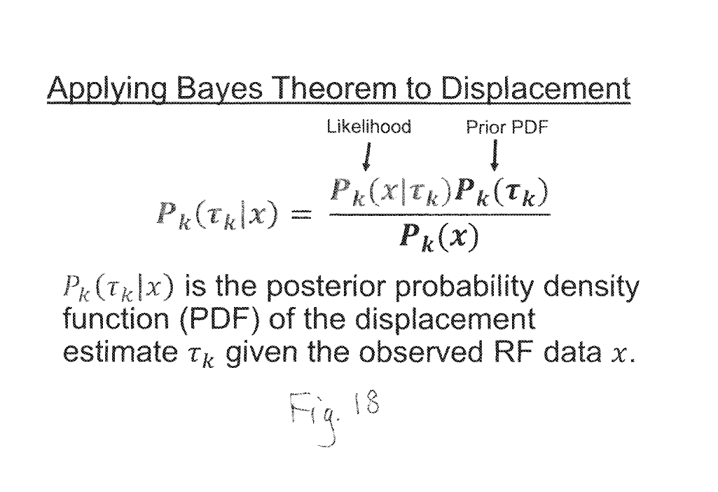

FIG. 18 is a diagram illustrating application of Bayes Theorem to Displacement. Shown is the probability of the displacement estimate, given an observed RF data. Prior posterior probability density function (PDF) is a spatial constraint rather than temporal.

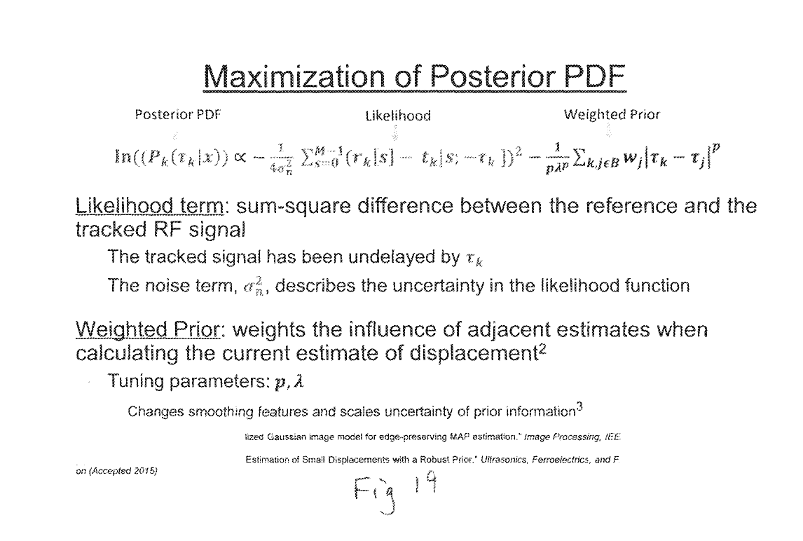

FIG. 19 is a diagram illustrating maximization of posterior PDF. The Likelihood function describes the likelihood of observing the tracked signal given a reference signal. The track signal has been delayed by −Tau(k). P=2, Gaussian. Lamda=standard deviation of the Gaussian or prior shape.

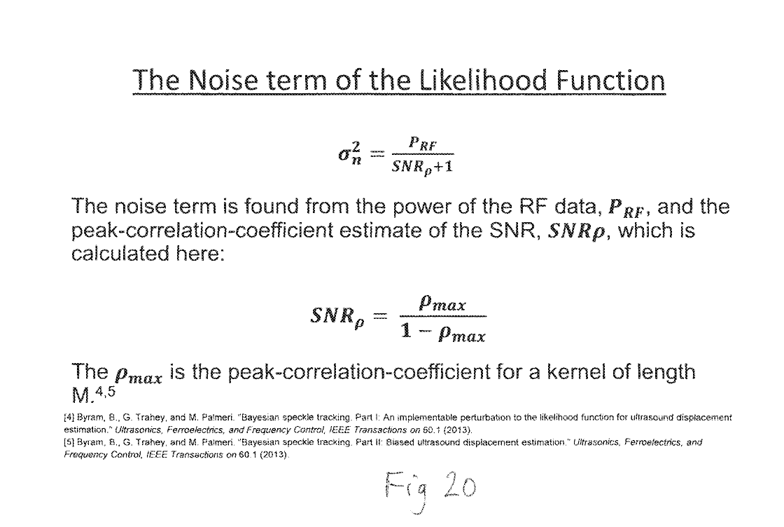

FIG. 20 is a diagram illustrating the noise term of the likelihood function. Uncertainty is derived from the likelihood function. It describes the local quality of the rf signal.

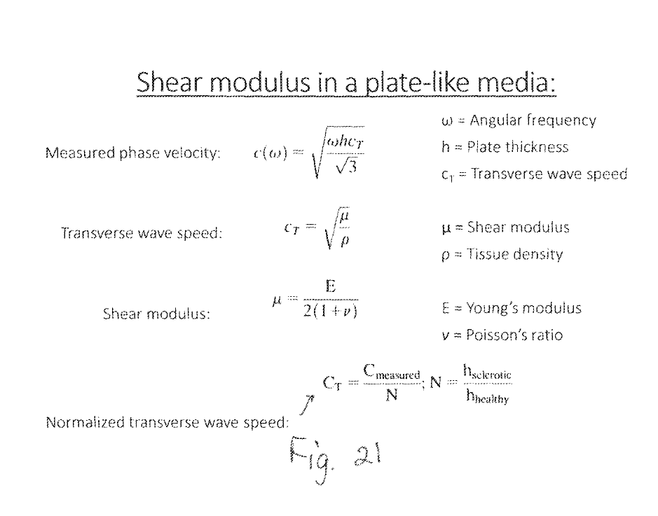

FIG. 21 is a diagram illustrating shear modulus in a plate-like media. Thickness introduces a frequency dependence of the transverse wave, and a Lamb wave model is used to convert the measured wave propagation speed to shear moduli. In typical incompressive, homogeneous media, Poisson's ratio is approximated to 0.5, but here the dermis is a thin layer (free on one side and bounded on the other) which makes the boundary conditions more complicated. Thus, SWS is reported as an indicator of stiffness.

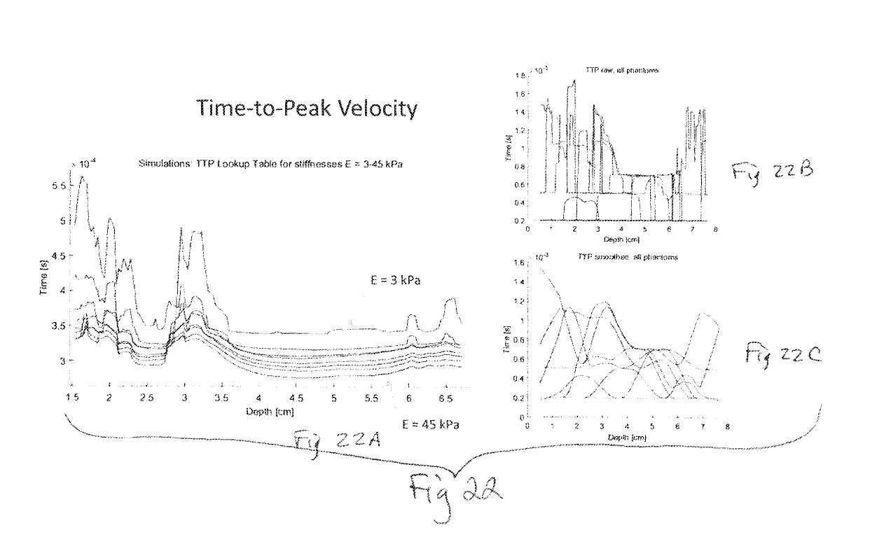

FIG. 22 is a plot comparison illustrating time-to-peak velocity including simulations of phantoms using a look-up table, and with raw and smooth filtering applied.

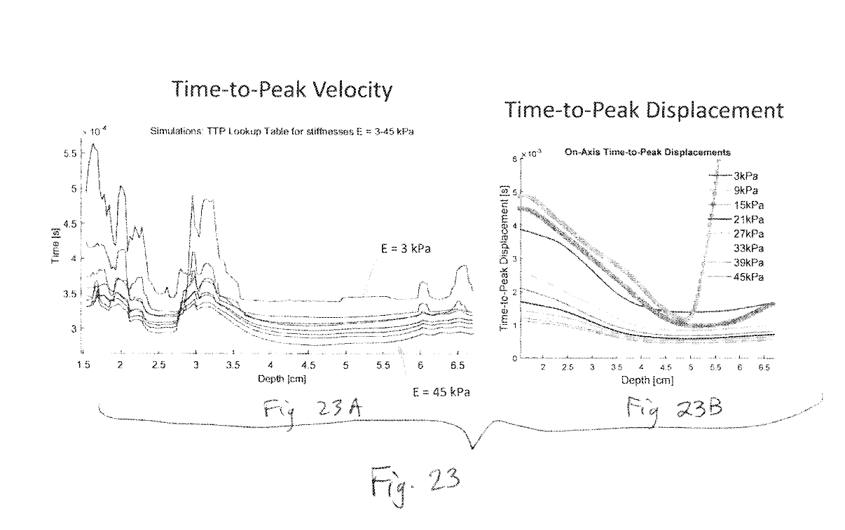

FIG. 23 is a diagram illustrating look-up table simulation in FIG. 22 in comparison with an on-axis time-to-peak displacement.

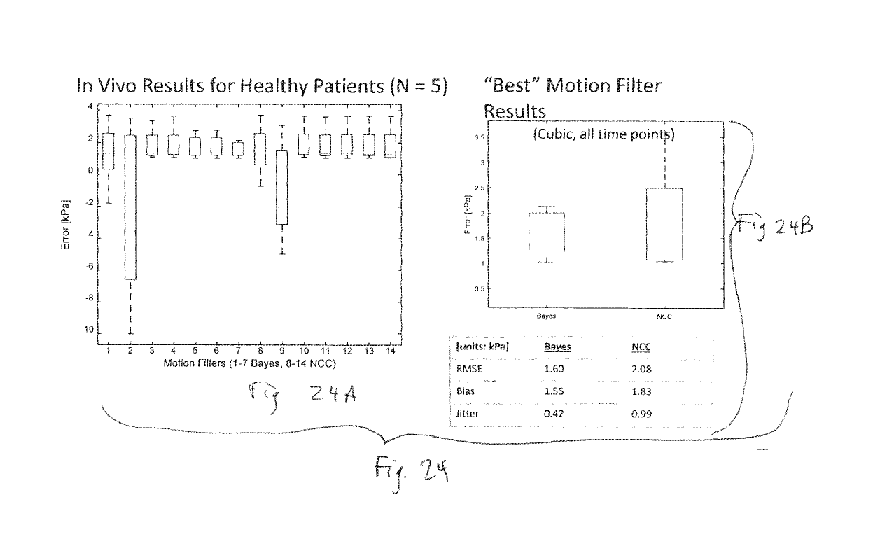

FIG. 24 is a plot comparison illustrating results of Bayes vs. NCC estimations with motion filtering included.

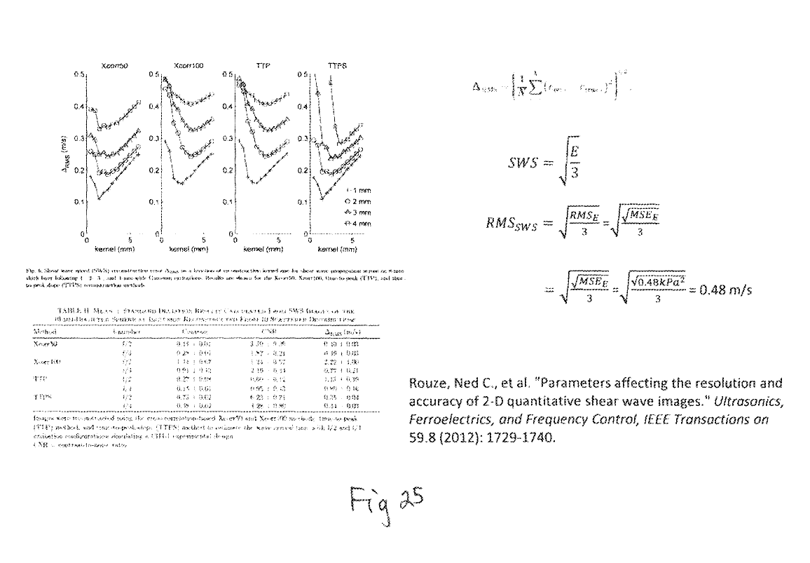

FIG. 25 is a diagram illustrating how error from an embodiment of an on-axis method is within the same range as SWEI error.

Embodiments are directed to an ultrasound device for calculating an estimated tissue stiffness based on peak on-axis tissue displacement propagating along the axis of an acoustic radiation force (ARF) excitation region. The device comprises: a transducer that outputs acoustic radiation to induce ARF along the axis to displace the tissue; a receiver that receives, along the axis, acoustic radiation reflected by the displaced tissue and which is based on the ARF-induced tissue displacement, wherein the reflected acoustic radiation corresponds to a tissue response to the tissue displacement; and an analysis system. The analysis system comprises: an adaptive displacement estimator that estimates the displacement of the tissue at a plurality of depths per each select location along the axis; a timing system that determines (or identifies) a time for the tissue to achieve peak displacement; and a stiffness estimator that determines the estimated stiffness of the tissue based on the time for the tissue to achieve the peak displacement. This procedure will estimate the on-axis displacements at all depths simultaneously. That is, scanning is performed on one on-axis line at all depths through time. Thus, data at this one location is acquired repeatedly.

In an embodiment, the timing system determines (or identifies) the time for the tissue to achieve peak displacement using the estimated displacement of the tissue at the plurality of depths along the axis. The time-to-peak displacement is found after the adaptive displacement estimator.

In an embodiment, the adaptive displacement estimator determines a probability of the estimated displacement given the tissue response. That is, the tissue response encoded within the acoustic radiation force data is measured. The adaptive displacement estimator may determine the sum square difference between the tissue response and a prior acoustic radiation reference signal weighted by local estimates of displacement estimation quality and prior information about acoustic radiation displacement behavior of the tissue at the plurality of depths along the axis. The weighting of adjacent estimates are still on-axis estimates. They use information from the displacement at the previous depth to weight the displacement at the current depth being estimated.

In an embodiment, the adaptive displacement estimator is a Bayesian displacement estimator.

In an embodiment, the time determined (or identified) by the timing system is an estimated time.

In an embodiment, the stiffness estimator comprises a lookup table (e.g., of interpolated type, using known stiffness values), wherein the estimated stiffness of the tissue is determined using the lookup table.

In an embodiment, the estimated stiffness of the tissue is determined by the stiffness estimator via a simulation or measurements from calibrated phantoms.

In an embodiment, the tissue is selected from the group consisting of liver, spleen, skin, heart, brain, muscle, and bone. Other tissue may of course be contemplated within the scope of the present invention. For example, any tissue that can be reached with ultrasound is plausible and may further include, for example, tissues reached via laparoscopic procedures, transesophageal devices, intracolon devices, etc.

In an embodiment, the analysis system further comprises a motion filter that filters motion of the displacement of the tissue not due to the outputted acoustic radiation. The adaptive displacement estimator gives us displacements of tissue motion that includes radiation force motion. It is motion filtered afterwards.

In an embodiment, the receiver is contained within the transducer.

In an embodiment, the ultrasound device comprises only a single transducer.

In an embodiment, the outputted acoustic radiation comprises long acoustic pulses and wherein the reflected acoustic radiation comprises short acoustic pulses which are shorter than the long acoustic pulses.

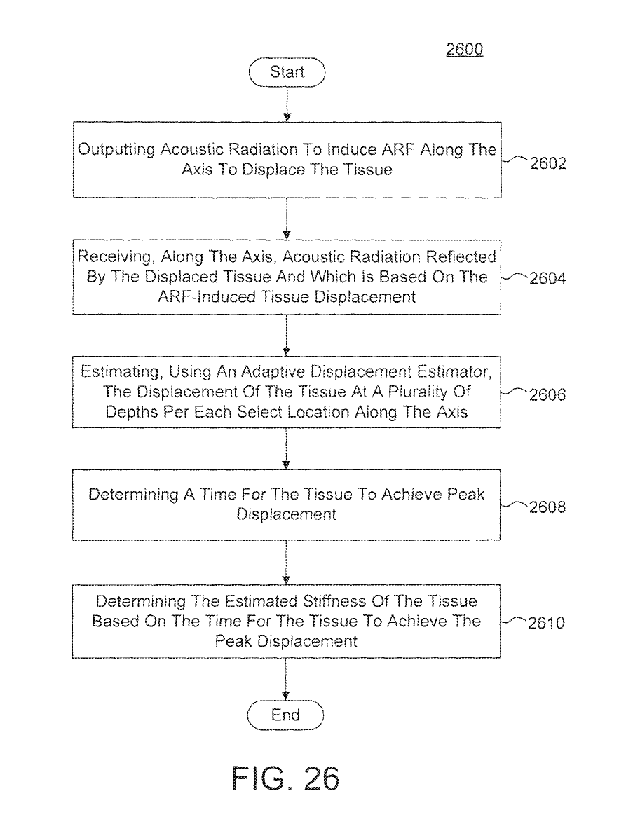

With reference to FIG. 26, shown is a flowchart illustrating an embodiment of a method 2600 for calculating an estimated tissue stiffness based on peak on-axis tissue displacement propagating along the axis of an acoustic radiation force (ARF) excitation region. The method 2600 includes: outputting acoustic radiation to induce ARF along the axis to displace the tissue (block 2602); receiving, along the axis, acoustic radiation reflected by the displaced tissue and which is based on the ARF-induced tissue displacement, wherein the reflected acoustic radiation corresponds to a tissue response to the tissue displacement (block 2604); estimating, using an adaptive displacement estimator, the displacement of the tissue at a plurality of depths per each select location along the axis (block 2606); determining a time for the tissue to achieve peak displacement (block 2608); and determining the estimated stiffness of the tissue based on the time for the tissue to achieve the peak displacement (block 2610).

In an embodiment, the determining of the time for the tissue to achieve peak displacement uses the estimated displacement of the tissue at the plurality of depths along the axis from the estimating step.

In an embodiment, the estimating step comprises determining, using the adaptive displacement estimator, a probability of the estimated displacement given the tissue response. The estimating step may further comprise determining, using the adaptive displacement estimator, the sum square difference between the tissue response and a prior acoustic radiation reference signal weighted by local estimates of displacement estimation quality and prior information about acoustic radiation displacement behavior of the tissue at the plurality of depths along the axis.

In an embodiment, the adaptive displacement estimator is a Bayesian displacement estimator.

In an embodiment, the time determined by the determining the time step is an estimated time.

In an embodiment, the determining of the estimated stiffness of the tissue comprises using a lookup table.

In an embodiment, the determining of the estimated stiffness of the tissue comprises using a simulation or measurements from calibrated phantoms.

In an embodiment, the tissue is selected from the group consisting of liver, spleen, skin, heart, brain, muscle, and bone. Other tissue may of course be contemplated within the scope of the present invention. For example, any tissue that can be reached with ultrasound is plausible and may further include, for example, tissues reached via laparoscopic procedures, transesophageal devices, intracolon devices, etc.

In an embodiment, the method further comprises filtering motion of the displacement of the tissue not due to the outputted acoustic radiation.

In an embodiment, the outputting and receiving steps are performed by a single transducer.

In an embodiment, the outputted acoustic radiation comprises long acoustic pulses and wherein the reflected acoustic radiation comprises short acoustic pulses which are shorter than the long acoustic pulses.

In any of the embodiments described herein, the transducer may apply the output acoustic radiation to tissue (i.e., to generate acoustic radiation force induced displacements) at focal depths below the tissue surface down to depths of about 8-10 cm or even deeper dependent on adequate generation of acoustic radiation force. Using the Bayesian displacement estimator, we can measure ARF responses with a peak displacement as low as about 0.1 μm. However, this is dependent on, for example, the frequency of the transducer. At higher frequencies, displacements less than this could be measured. Thus, focal depths outside of this range may be possible. Alternatively, the focal depth may be on the tissue outer surface.

Although embodiments are described above with reference to an ultrasound device and an ultrasound transducer outputting the acoustic radiation (force) excitation to displace the tissue in order to achieve a tissue response therefrom, other external sources of transient mechanical excitation (such as a Fibroscan®-like punch or other external vibration source) directed at the target tissue along the axis may be contemplated in any of the above embodiments. Thus, the on-axis displacements could be measured using modalities other than an ultrasound device such as those used for optical coherence tomography (OCT). These alternatives may therefore utilize the advantages of the configurations and embodiments described above.

Features in any of the embodiments described in this disclosure may be employed in combination with features in other embodiments described herein, such combinations are considered to be within the spirit and scope of the present invention.

The contemplated modifications and variations specifically mentioned in this disclosure are considered to be within the spirit and scope of the present invention.

Those of ordinary skill in the art will recognize that various modifications and variations may be made to the embodiments described in this disclosure without departing from the spirit and scope of the present invention. It is therefore to be understood that the present invention is not limited to the particular embodiments disclosed herein, but it is intended to cover such modifications and variations as defined by the following claims.

1. An ultrasound device for calculating an estimated tissue stiffness based on peak on-axis tissue displacement propagating along the axis of an acoustic radiation force (ARF) excitation region, the device comprising:
a transducer that outputs acoustic radiation to induce ARF along the axis to displace the tissue;
a receiver that receives, along the axis, acoustic radiation reflected by the displaced tissue and which is based on the ARF-induced tissue displacement, wherein the reflected acoustic radiation corresponds to a tissue response to the tissue displacement; and
an analysis system comprising:
an adaptive displacement estimator that estimates the displacement of the tissue at a plurality of depths per each select location along the axis;
a timing system that determines a time for the tissue to achieve peak displacement; and
a stiffness estimator that determines the estimated stiffness of the tissue based on the time for the tissue to achieve the peak displacement.

2. The ultrasound device of claim 1, wherein the timing system determines the time for the tissue to achieve peak displacement using the estimated displacement of the tissue at the plurality of depths along the axis.

3. The ultrasound device of claim 1, wherein the adaptive displacement estimator determines a probability of the estimated displacement given the tissue response.

4. The ultrasound device of claim 3, wherein the adaptive displacement estimator determines the sum square difference between the tissue response and a prior acoustic radiation reference signal weighted by local estimates of displacement estimation quality and prior information about ARF displacement behavior of the tissue at the plurality of depths along the axis.

5. The ultrasound device of claim 1, wherein the adaptive displacement estimator is a Bayesian displacement estimator.

6. The ultrasound device of claim 1, wherein the time determined by the timing system is an estimated time.

7. The ultrasound device of claim 1, wherein the stiffness estimator comprises a lookup table, wherein the estimated stiffness of the tissue is determined using the lookup table.

8. The ultrasound device of claim 1, wherein the estimated stiffness of the tissue is determined by the stiffness estimator via a simulation or measurements from calibrated phantoms.

9. The ultrasound device of claim 1, wherein the tissue is selected from the group consisting of liver, spleen, skin, heart, brain, muscle, and bone.

10. The ultrasound device of claim 1, wherein the analysis system further comprises a motion filter that filters motion of the displacement of the tissue not due to the outputted acoustic radiation.

11. The ultrasound device of claim 1, wherein the receiver is contained within the transducer.

12. The ultrasound device of claim 1, comprising only a single transducer.

13. The ultrasound device of claim 1, wherein the outputted acoustic radiation comprises long acoustic pulses and wherein the reflected acoustic radiation comprises short acoustic pulses which are shorter than the long acoustic pulses.

14. A method for calculating an estimated tissue stiffness based on peak on-axis tissue displacement propagating along the axis of an acoustic radiation force (ARF) excitation region, the method comprising:
outputting acoustic radiation to induce ARF along the axis to displace the tissue;
receiving, along the axis, acoustic radiation reflected by the displaced tissue and which is based on the ARF-induced tissue displacement, wherein the reflected acoustic radiation corresponds to a tissue response to the tissue displacement;
estimating, using an adaptive displacement estimator, the displacement of the tissue at a plurality of depths per each select location along the axis;
determining a time for the tissue to achieve peak displacement; and
determining the estimated stiffness of the tissue based on the time for the tissue to achieve the peak displacement.

15. The method of claim 14, wherein the determining of the time for the tissue to achieve peak displacement uses the estimated displacement of the tissue at the plurality of depths along the axis from the estimating step.

16. The method of claim 14, wherein the estimating step comprises determining, using the adaptive displacement estimator, a probability of the estimated displacement given the tissue response.

17. The method of claim 16, wherein the estimating step further comprises determining, using the adaptive displacement estimator, the sum square difference between the tissue response and a prior acoustic radiation reference signal weighted by local estimates of displacement estimation quality and prior information about ARF displacement behavior of the tissue at the plurality of depths along the axis.

18. The method of claim 14, wherein the adaptive displacement estimator is a Bayesian displacement estimator.

19. The method of claim 14, wherein the time determined by the determining the time step is an estimated time.

20. The method of claim 14, wherein the determining of the estimated stiffness of the tissue comprises using a lookup table.

21. The method of claim 14, wherein the determining of the estimated stiffness of the tissue comprises using a simulation or measurements from calibrated phantoms.

22. The method of claim 14, wherein the tissue is selected from the group consisting of liver, spleen, skin, heart, brain, muscle, and bone.

23. The method of claim 14, further comprising filtering motion of the displacement of the tissue not due to the outputted acoustic radiation.

24. The method of claim 14, wherein the outputting and receiving steps are performed by a single transducer.

25. The ultrasound device of claim 14, wherein the outputted acoustic radiation comprises long acoustic pulses and wherein the reflected acoustic radiation comprises short acoustic pulses which are shorter than the long acoustic pulses.
